(19) United States
(12) Patent Application Publication
(10) Pub. No.: US 2022/0387678 A1
(43) Pub. Date: Dec. 8, 2022

(54) SCALP CUPPING DEVICE, ELECTRONIC CUPPING DEVICE SYSTEM HAVING SAME, AND USE METHOD THEREFOR

(71) Applicant: Hyun Ja PARK, Gwangmyeong-si (KR)

(72) Inventor: Sang Jun PARK, Gunpo-si (KR)

(73) Assignee: Hyun Ja Park, Gwangmyeong-si (KR)

(21) Appl. No.: 17/641,951

(22) PCT Filed: Oct. 1, 2019
(86) PCT No.: PCT/KR2019/012817

§ 371 Date: Aug. 26, 2022

(30) Foreign Application Priority Data
Sep. 10, 2019 (KR) 10-2019-0112022

(51) Int. Cl. A61M 1/08; A61M 1/00

(52) U.S. Cl. CPC A61M 1/08; A61M 1/80; A61M 2205/0216; A61M 2205/3341; A61M 2205/3344

(57) ABSTRACT

The present invention relates to a scalp cupping device, an electronic cupping device system having same, and a use method therefor. The scalp cupping device, according to the present invention, for discharging body waste by using a negative-pressure load obtaining suction force from the removal of the inner air thereof can comprise: a negative pressure-controlling main cup having a negative pressure adjustment means provided at the upper side thereof so as to apply a negative-pressure load to the inside thereof; and a body contact auxiliary cup which is inserted into and coupled to the opened lower portion of the main cup, and which has, on the circumference of the bottom opening thereof, a contact portion for coming in contact with the body.

### TECHNICAL FIELD

The present invention relates to a scalp cupping device, an electronic cupping device system having the same, and a use method therefor, and more specifically, to a scalp cupping device includes an attachment suction member made of a heterogeneous silicone material and inserted into and coupled to a main body of the cupping device, so that the cupping therapy can be performed on various areas where it is difficult to use a cupping device, including the scalp and joint areas, as well as, allows a user to easily adjust the negative pressure inside each cupping device even when using multiple cupping devices, and automatically blocks an exhaust pipe of the cupping device causing a loss of negative pressure from among the multiple cupping devices so as to enable suitable control for the negative pressure loss of the cupping device, thereby providing the stable cupping therapy, an electronic cupping device system having the same, and a use method therefor.

### BACKGROUND ART

In general, a cupping apparatus is an oriental medicine treatment apparatus used for vacuum lifeblood treatment, blood purification and improvement of physical constitution. Cupping therapy using such a cupping apparatus is an oriental medicine treatment method in which a cupping apparatus (cup) with an opening in one side is put on the skin on an acupuncture point, and air in the cupping apparatus is drawn out from the interior space of the cupping apparatus to maintain a negative pressure (vacuum) state in the interior of the cupping apparatus so that the cupping apparatus is fixed onto the skin while the skin is inhaled into the interior of the cupping apparatus by the negative pressure, and a bodily waste, toxin, or bad blood is extracted through pores.

The cupping therapy induces smooth metabolism of the body and blood purification, expands capillaries, promotes blood circulation, and discharges wastes and toxins to the outside of the body to treat the affected part to be treated.

As a cupping apparatus according to the traditional method, a cup-shaped ceramic product is mainly used. The cupping apparatus which is the ceramic product has drawbacks in that it is broken due to carelessness during use and it is inconvenient for use, such as setting alcohol on fire.

Recently, by improving such drawbacks, a cupping apparatus having an air suction means, unlike the traditional cupping therapy, has been used.

A cupping apparatus having an air suction means according to the prior art may include a check valve, an air inside the cupping apparatus is drawn out using an air suction pump to generate a negative pressure inside the cupping apparatus, thereby attaching the cupping apparatus onto the affected part and at the same time removing bodily wastes or toxins through pores by means of the suction force.

However, in the cupping apparatus according to the prior art, an open lower portion of a cup is made of a rigid material, such as synthetic resin, and thus the cupping apparatus has a problem in that it is difficult to use in joint areas having uneven surfaces, as well as skin areas, areas with thin muscles, or hairy areas.

Particularly, when cupping therapy is performed on the head, it is not easy to maintain a negative-pressure load inside the cupping apparatus due to the thickness of the hair and the gap between adjacent hairs. In order to solve such problems, if hair is removed from contact areas between the head and the cupping apparatuses, several hair-removed areas may make a recipient feel uncomfortable in social life and may result in a tendency to avoid treatment procedures.

In addition, when cupping therapy is performed using several cupping apparatus according to the prior art, negative pressure loss is not independently controlled for each cupping apparatus, and thus if one cupping apparatus is separated from a treatment site and a large amount of air is introduced thereinto, a vacuum pump cannot function properly so that all other negative pressures cannot be maintained.

Therefore, there is an urgent need for a practically applicable technology that can apply the cupping therapy on an area including a head part of the human body where it is difficult to use a cupping apparatus, as well as allows a user to easily adjust internal negative pressure of a cupping apparatus even when using a number of cupping apparatuses, thereby providing the stable cupping therapy.

### Technical Problem

The present invention has been devised to solve the above problems, and an object of the present invention is to provide a scalp cupping device, an electronic cupping device system having the same, and a method of using the cupping device, wherein the scalp cupping device includes an attachment suction member made of a heterogeneous silicone material and inserted into and coupled to a main body of the cupping device, so that the cupping therapy can be performed on various areas where it is difficult to use a cupping device, including the scalp and joint areas, as well as, allows a user to easily adjust the negative pressure inside each cupping device even when using multiple cupping devices, and automatically blocks an exhaust pipe of the cupping device causing a loss of negative pressure from among the multiple cupping devices so as to enable suitable control for the negative pressure loss of the cupping device, thereby providing the stable cupping therapy.

### Technical Solution

A scalp cupping device, according to an embodiment of the present invention, for discharging body waste by using a negative-pressure load obtaining suction force from the removal of the inner air thereof may include: a negative pressure-controlling main cup having a negative pressure adjustment means provided at the upper side thereof so as to apply a negative-pressure load to the inside thereof; and a body contact auxiliary cup which is inserted into and coupled to the opened lower portion of the main cup, and which has, on the circumference of the bottom opening thereof, a contact portion for coming in contact with the body.

In the scalp cupping device according to an embodiment of the present invention, the negative pressure-controlling main cup may be formed in the shape of a circular cup made of a plastic material with an open lower portion, and the body contact auxiliary cup may be formed in the shape of a circular cup made of a relatively flexible material compared to the main cup so as to seal the contact portion when inserted into and coupled to the negative pressure-controlling main cup.

The body contact auxiliary cup may have a through hole formed in the upper central portion thereof to transmit the negative-pressure load applied to the main cup and may have an outwardly protruding circumferential periphery of the bottom opening to come in contact with the body.

The body contact auxiliary cup may include a coupling part which is formed on the upper side thereof and made of a medium-hardness silicone material to prevent the inflow of external air by being in soft contact with the contact portion when coupled to the main cup; a suction part which is formed on the lower side thereof and which is made of a material having a relatively low hardness compared to the coupling part and is formed by overlaying or connecting a low-hardness silicone material so as to be securely attached without gaps even in contact with the scalp and hair; and a holder which is formed in a hook shape between the coupling part and the suction part and made of a medium-hardness silicone material having a relatively high hardness compared to the suction part so as not to be sucked into the main cup.

The negative pressure adjustment means provided in the main cup may include an exhaust pipe for discharging the inner air to generate a negative pressure; an exhaust valve for discharging the inner air or blocking the air from flowing into the inside of the main cup; and a negative release button for releasing the negative pressure load applied to the inside of the main cup, and the negative pressure adjustment means may be provided between the main cup and a suction hose or between the suction hose and a suction pipe.

The negative pressure release button may further include a button press spring device (not shown) and a rotational latch (not shown) to keep the negative pressure release button always in a negative pressure release state in order to prevent damage caused by strong air pressure acting on the main cup to eyes, ears, soft skin, etc. of children or the weak.

The exhaust pipe may include a blocking plate spring vertically formed therein and a blocking plate formed in a disk shape and freely positioned between a lower end of the blocking plate spring and an inlet hole formed on an upper surface of the main cup, and the exhaust valve may include an exhaust blocking release button capable of moving the blocking plate in a downward vertical direction on the upper surface thereof in an area adjacent to the exhaust pipe.

The blocking plate may usually maintain an intermediate position at which it does not block either up or down directions inside the exhaust pipe, and may move up and down according to an air speed, wherein, when moving upward, the blocking plate pushes the blocking plate spring and seals the bottom surface of the exhaust pipe to block a loss of negative pressure, whereas by the speed of air introduced in a small amount between hairs, the blocking plate spring cannot be pushed and hence the bottom surface of the exhaust pipe is not sealed. In addition, when moving downward, the blocking plate may seal an inlet hole formed on an upper surface of the main cup to maintain a vacuum.

A blocking state in an exhaust direction may be released using the exhaust blocking release button, and a blocking state in an inflow direction may be released by using the negative pressure release button or by a suction operation of a vacuum pump.

An electronic cupping device system according to another embodiment of the present invention may include: a plurality of scalp cupping devices, each of which includes a negative pressure-controlling main cup made of a plastic material and having a negative pressure adjustment means composed of an exhaust pipe, an exhaust valve, and a negative pressure release button provided on an upper side thereof so as to apply a negative-pressure load to the inside thereof, and a body contact auxiliary cup inserted into and coupled to an opened lower portion of the main cup and made of a silicone material relatively flexible compared to the main cup to increase a sealing force when in contact with the body; a negative pressure control means configured to individually control the negative-pressure load applied to the inside of the plurality of scalp cupping devices; and a power control unit configured to control the power supplied to the negative pressure control means.

The negative pressure control means may include a control unit having a plurality of timer switches in a one-to-one correspondence to the plurality of scalp cupping devices; a plurality of suction pipes connected in a one-to-one correspondence between the timer switches of the control unit and suction hoses connected to the exhaust pipes of the plurality of scalp cupping devices; a plurality of on/off operation units each of which is disposed between each exhaust pipe constituting the plurality of scalp cupping devices and the suction pipe to control an on/off operation of the timer switch of the control unit with a remote button; and a plurality of pressure control devices connected in a one-to-one correspondence between the plurality of suction pipes and the plurality of on/off operation units so as to check and set a pressure value applied to each of the plurality of scalp cupping devices.

The negative pressure control means may each automatically stop the suction operation when the pressure applied to each of the plurality of scalp cupping devices exceeds a specific range.

A method, according to still another embodiment of the present invention, of using an electronic cupping device system that uses a plurality of scalp cupping devices each of which includes a negative pressure-controlling main cup made of a plastic material and a body contact auxiliary cup inserted into and coupled to an open lower portion of the main cup and made of a silicone material relatively flexible compared to the main cup to increase the sealing force when in contact with the body, may include: selecting at least one cupping device channel to be operated from among the plurality of scalp cupping devices; setting an operation time of the selected cupping device channel by using a timer switch of a control unit provided in a negative pressure control means; setting a pressure of the selected cupping device channel using a pressure control device provided in the negative pressure control means; checking a button of an on/off operation unit provided in the negative pressure control means and, when an operation is in on state, checking whether a pressure gauge provided in the pressure control device indicates improper pressure; when there is a sudden loss of negative pressure by checking whether the pressure gauge provided in the pressure control device indicates improper pressure, blocking an exhaust valve of the selected cupping device channel to automatically stop a suction operation; and when there is no sudden loss of negative pressure by checking whether the pressure gauge provided in the pressure control device indicates improper pressure, operating in a reverse mode and resolving issues for stopping the suction operation by reconnecting a disconnected suction hose to resume a cupping procedure.

Additional features of the invention will be set forth in the description which follows, and in part will be apparent from the description, or may be learned by practice of the invention.

### Advantageous Effects

As described above, according to the present invention, a scalp cupping device, an electronic cupping device system using the same, and a method of using the cupping device are provided. The scalp cupping device includes an attachment suction member made of a heterogeneous silicone material and inserted into and coupled to a main body of the cupping device so that the cupping therapy can be performed on various areas where it is difficult to use a cupping device, including the scalp and joint areas. In addition, a user can easily adjust the negative pressure inside each cupping device even when using multiple cupping devices, and the exhaust pipe of the cupping device causing a loss of negative pressure from among the multiple cupping devices is automatically blocked to enable suitable control for the negative pressure loss of the cupping device, thereby providing the stable cupping therapy.

### DESCRIPTION OF DRAWINGS

FIG. 1 is a schematic diagram illustrating a configuration of a scalp cupping device according to an embodiment of the present invention.

FIG. 2 is a cross-sectional view illustrating a main cup and an auxiliary cup coupled to each other according to an embodiment of the present invention.

FIG. 3 is a diagram for schematically explaining the operating principle of the scalp cupping device shown in FIGS. 1 and 2.

FIG. 4 is a diagram for schematically explaining an electronic cupping system having a scalp cupping device according to another embodiment of the present invention.

FIGS. 5 and 6 are flowchart for schematically explaining a method of using an electrical cupping system according to another embodiment of the present invention.

### MODE FOR INVENTION

The descriptions of the invention are only exemplary embodiments of the invention. The scope of the invention is not to be construed as being limited to the exemplary embodiments described herein. That is, while the invention is susceptible to various modifications and alternative forms, it should be understood that the invention is to cover all modifications, equivalents, and alternatives falling within the spirit and scope of the invention.

Meanwhile, it should be understood that the terms used herein are defined as follows.

It will be understood that, although the terms first, second, etc may be used herein to describe various elements, these elements should not be limited by these terms. These terms are only used to distinguish one element from another. For example, a first element could be termed a second element, and, similarly, a second element could be termed a first element, without departing from the scope of the present invention.

It will be understood that when an element is referred to as being “on top” or “on” another element, it can be directly on the other element or intervening elements may be present. On the other hand, when an element is referred to as being “in contact with” another element, there are no intervening elements present. Meanwhile, other words used to describe the relationship between elements should be interpreted in a like fashion (i.e., “between” versus “directly between,” “adjacent” versus “directly adjacent,” etc.).

Singular forms “a”, “an” and “the” in the present disclosure are intended to include the plural forms as well, unless the context clearly indicates otherwise. It will be further understood that terms such as “including” or “having,” etc., are intended to indicate the existence of the features, numbers, operations, actions, components, parts, or combinations thereof disclosed in the specification, and are not intended to preclude the possibility that one or more other features, numbers, operations, actions, components, parts, or combinations thereof may exist or may be added.

Identification letters (e.g., a, b, c, etc.) in respective steps or operations are used for the sake of explanation and do not describe any particular order. The respective operations may be changed from a mentioned order unless specifically mentioned in context. Namely, respective steps may be performed in the same order as described, may be substantially simultaneously performed, or may be performed in reverse order.

The terms used in the present application are merely used to describe particular embodiments, and are not intended to limit the present invention. Unless otherwise defined, all terms used herein, including technical or scientific terms, have the same meanings as those generally understood by those with ordinary knowledge in the field of art to which the present invention belongs. Such terms as those defined in a generally used dictionary are to be interpreted to have the meanings equal to the contextual meanings in the relevant field of art, and are not to be interpreted to have ideal or excessively formal meanings unless clearly defined in the present application.

A cupping device according to the present invention relates to a cupping device for discharging a bodily waste by using a negative-pressure load obtaining suction force from the removal of the inner air thereof.

Hereinafter, embodiments of the present invention will be described with reference to the accompanying drawings.

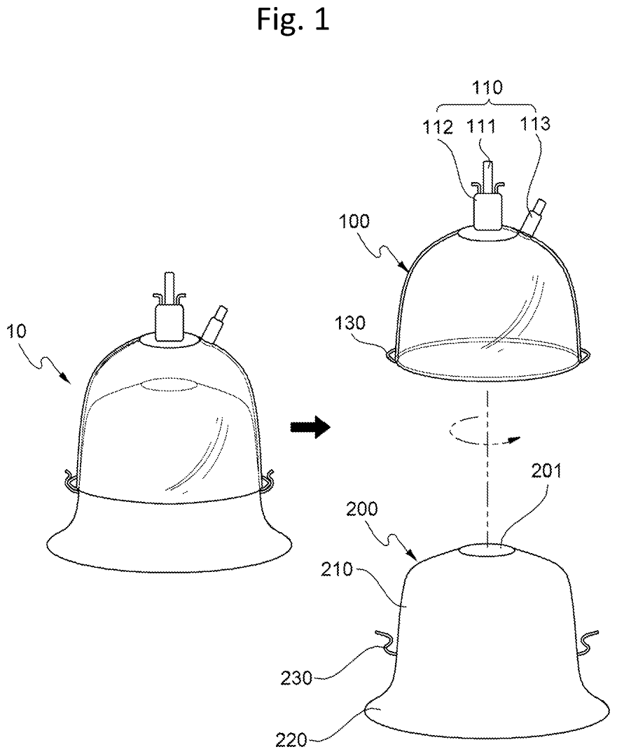

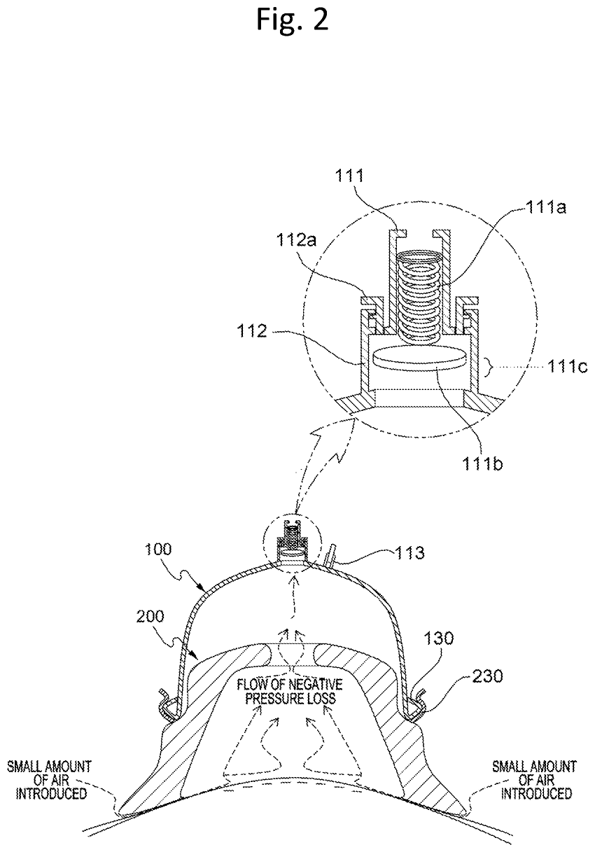

As shown in FIGS. 1 and 2, a scalp cupping device according to an embodiment of the present invention may include a negative pressure-controlling main cup 100 and a body contact auxiliary cup 200.

More specifically, the negative pressure-controlling main cup 100 may have a negative pressure adjustment means 110 provided at the upper side thereof to apply a negative-pressure load to the inside thereof.

In this case, the negative pressure-controlling main cup 100 may be formed in the shape of a circular cup made of a transparent plastic material with an open lower portion, as shown in the drawing.

In an embodiment of the present invention, the negative pressure adjustment means 110 provided in the main cup includes an exhaust pipe 111, an exhaust valve 112, and a negative pressure release button 113 provided on the upper side thereof, and the negative pressure adjustment means 110 may be disposed between the main cup 100 and a suction hose 301, or between the suction hose 301 and a suction pipe 320, and may have a holder engaging part 130 on the outer surface of a lower end to be engaged with a holder 230 of the auxiliary cup 200, which will be described below.

Here, the exhaust pipe 111 may generate a negative pressure by discharging the air inside the main cup, and the exhaust valve 112 may discharge the air inside the main cup or block the air from flowing into the inside the main cup.

More specifically, as shown in FIG. 2, the exhaust pipe 111 may include a blocking plate spring 111a vertically formed therein and a blocking plate 111b formed in a disk shape at the end of the blocking plate spring 111a.

In this case, an exhaust blocking release button 112a capable of moving the blocking plate 111b in a downward vertical direction may be formed on the upper surface of the exhaust valve 112 in an area adjacent to the exhaust pipe 111.

When described in more detail with reference to the drawings, the blocking plate 111b usually maintains an intermediate position 111c at which it does not block either up or down directions inside the exhaust pipe 111 at the lower end of the blocking plate spring 111a. The blocking plate 111b moves up and down according to the air speed, wherein, when moving upward, the blocking plate 111b seals the bottom surface of the exhaust pipe 111 to block the exhaust direction, and when moving downward, the blocking plate 111b may seal an inlet hole formed on an upper surface of the main cup to maintain a vacuum.

Therefore, the blocking state in the exhaust direction may be released by using the exhaust blocking release button 112a, and the blocking state in the inflow direction may be released by using the negative pressure release button 113 to be described below or by a suction operation of a vacuum pump.

That is, the negative pressure release button 113 may release the negative-pressure load applied to the inside of the main cup.

In addition, when the main cup 100 and the auxiliary cup 200 are rotated to be coupled to each other while being stacked on each other, the holder engaging part 130 may be firmly coupled to the fixing member 230 of the auxiliary cup 200.

In an embodiment of the present invention, the body contact auxiliary cup 200 may be inserted into and coupled to an open lower portion of the main cup 100, and have, on the circumference of the bottom opening thereof, a contact portion for coming in contact with the body.

Here, the body contact auxiliary cup 200 may be formed in the shape of a circular cup made of a relatively flexible material compared to the main cup 100 so as to seal the contact portion when inserted into and coupled to the negative pressure-controlling main cup 100.

In addition, the body contact auxiliary cup 200, as shown in the drawing, may have a through hole 201 formed in the upper central portion thereof to transmit the negative-pressure load applied to the main cup 100, and may have an outwardly protruding circumferential periphery of the bottom opening to come in contact with the body.

Meanwhile, the body contact auxiliary cup 200 may include a coupling part 210, a suction part 220, and the holder 230.

More specifically, the coupling part 210 may be formed on the upper side, and may be made of a medium-hardness silicone material to prevent the inflow of external air by being in soft contact with the contact portion when coupled to the main cup 100.

In addition, the suction part 220 may be formed on the lower side, may be made of a material having a relatively low hardness compared to the coupling part 210, and may be formed by overlaying or connecting a low-hardness silicone material so that it can be securely attached without gaps even in contact with the scalp and hair.

In addition, the holder 230 may be formed in a hook shape between the coupling part 210 and the suction part 220, and may be made of a medium-hardness silicone material having a relatively high hardness compared to the suction part 220 so as not to be sucked into the main cup 100.

As such, the auxiliary cup 200 according to the embodiment of the present invention may be formed of a silicon material in an opaque state as a whole.

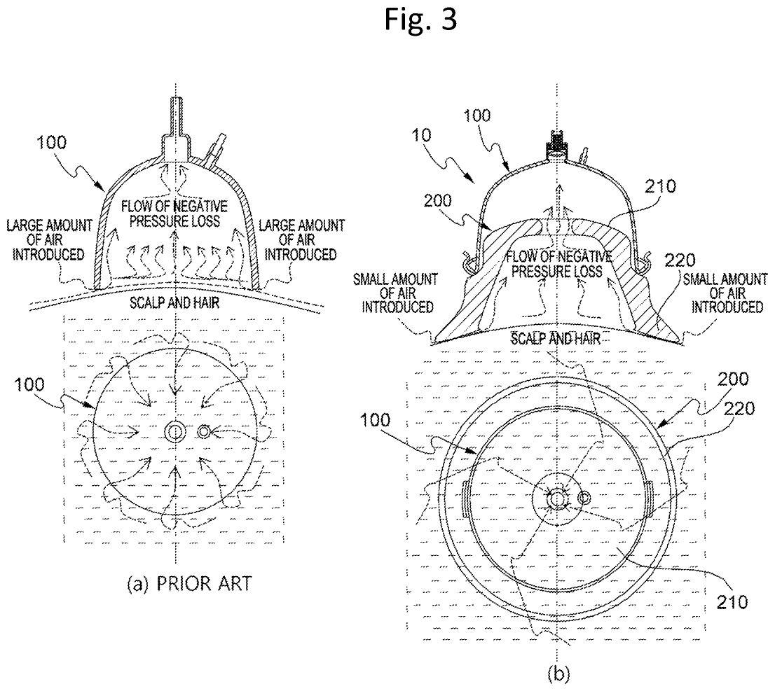

FIG. 3 is a diagram for schematically explaining the operating principle of the scalp cupping device shown in FIG. 1.

The operating principle of the scalp cupping device shown in FIG. 1 will be described below with reference to FIGS. 1 to 3.

First, as shown in the drawing (FIG. 3A), when the main cup 100 made of a plastic material is in direct contact with the scalp and hair, a large amount of air is introduced through the gap generated in the contact portion, which leads to an excessive loss of negative pressure.

On the contrary, when the auxiliary cup 200 made of a silicone material is used by being coupled to the main cup 100, even if there is the scalp and hair in the contact portion, only a small amount of air is introduced, thereby minimizing the inflow of the air and the loss of negative pressure.

On the other hand, according to the embodiment of the present invention, the main cup 100 may be formed of a plastic material, so that it can firmly support a high negative pressure in the cup that cannot be managed with a silicone material, and hence can maintain a desired pressure.

In addition, the auxiliary cup 200 may be formed of a silicone material having a hardness value of around 40 or more when the coupling part 210 in contact with the main cup 100 is measured with a shore hardness meter, so that, even when in contact with a plastic material, it can be in soft contact with the plastic material while preventing the inflow of external air. Here, the shore hardness meter preferably has a value close to 100 as the hardness of the material increases and close to 0 as the flexibility of the material increases.

In addition, the suction part 220 may be made of a silicone material having a value of around 15 based on a value measured with a shore hardness meter, so that the auxiliary cup 200 can be closely attached without gaps even when in direct contact with the scalp and hair.

Meanwhile, the holder formed on the coupling part 210 and the suction part 220 may be made of a medium-hardness silicone material having a hardness value similar to that of the coupling part 210, so that it can fix the auxiliary cup 200 so as not to be deeply sucked in the main cup 100 even under the high negative pressure in the main cup 100.

In this manner, the cupping device according to the embodiment of the present invention can be strongly attached to joint areas, such as knees, shoulders, elbows, etc., as well as the head of the human body having the scalp and hair, thereby facilitating the cupping procedure.

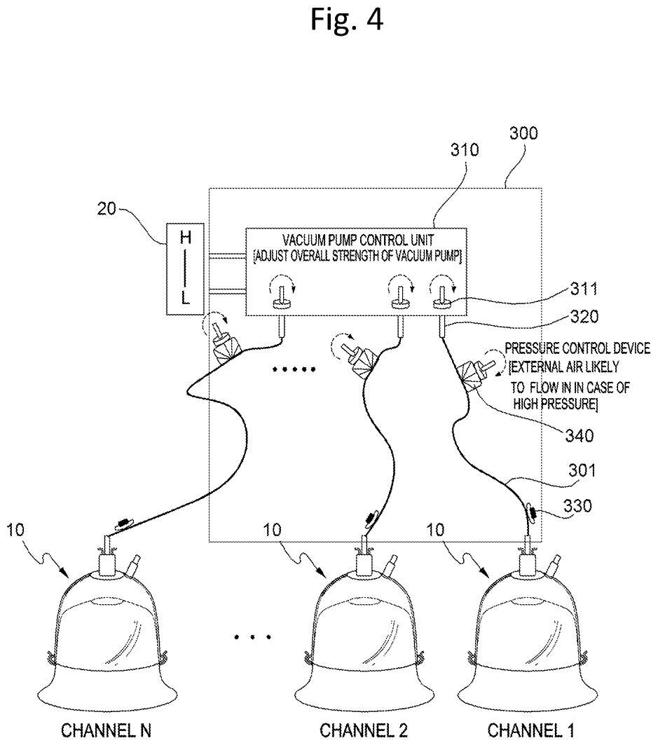

FIG. 4 is a diagram for schematically explaining an electronic cupping system having a scalp cupping device according to an embodiment of the present invention.

A detailed description of the same configuration as described above with reference to FIGS. 1 to 3 will be omitted.

As shown in the drawing, an electronic cupping system having a scalp cupping device according to an embodiment of the present invention may include a plurality of scalp cupping devices 10 composed of channels 1 to N, a negative pressure control means 300, and a power control unit 20.

In more detail, the plurality of scalp cupping devices 10 may each include a negative pressure-controlling main cup 100 having a negative pressure adjustment means 110 which is composed of an exhaust pipe 111, an exhaust valve 112, and a negative pressure release button 113 on an upper side thereof and may be provided between the main cup 100 and a suction hose 301, or between the suction hose 301 and a suction pipe 302, so as to apply a negative-pressure load to the inside of the main cup 100, and a body contact auxiliary cup 200 inserted into and coupled to the opened lower portion of the main cup and made of a silicone material relatively flexible compared to the main cup to increase the sealing force when in contact with the body.

In addition, the negative pressure control means 300 may individually control the negative-pressure load applied to the inside of the plurality of scalp cupping devices 10.

In addition, the power control unit 20 may control the power supplied to the negative pressure control means 300.

When described in more detail with reference to the drawings, the negative pressure control means 300 according to the embodiment of the present invention may include a control unit 310, the suction pipe 320, an on/off operation unit 330, and a pressure control device 340.

In more detail, the control unit 310 may include a plurality of timer switches 311 in a one-to-one correspondence to the plurality of scalp cupping devices 10.

Also, the suction pipe 320 may be provided in plural and connected in a one-to-one correspondence between the timer switches 311 of the control unit 310 and the suction hoses 301 connected to the exhaust pipes 111 of the plurality of scalp cupping devices 10.

In addition, the on/off operation unit 330 may be provided in plural and each disposed between each exhaust pipe 111 constituting the plurality of scalp cupping devices 10 and the suction pipe 320 to control the on/off operation of the timer switch 311 of the control unit 310 with a remote button.

In addition, the pressure control device 340 may be provided in plural and connected in a one-to-one correspondence between the plurality of suction pipes 320 and the plurality of on/off operation units 330 so as to check and set a pressure value applied to each of the plurality of scalp cupping devices 10.

On the other hand, according to an embodiment of the present invention, the negative pressure control means 300 may each automatically stop the suction operation when the pressure applied to each of the plurality of scalp cupping devices 10 exceeds a specific range.

As such, the electronic cupping system according to an embodiment of the present invention may restore the negative pressure in real time when even a very small amount of negative pressure is lost between the hairs, and may continuously suck the air out of the main cup 100 to maintain the desired pressure.

Further, due to the silicon material constituting the auxiliary cup 200 described above in FIG. 1, there is little or no air inflow into the cupping device by means of strong suction force, and therefore, there is almost no negative pressure loss inside the cupping device. Accordingly, the cupping procedure can be performed effectively and conveniently on areas that are difficult to perform the cupping procedure only using the cup made of plastic.

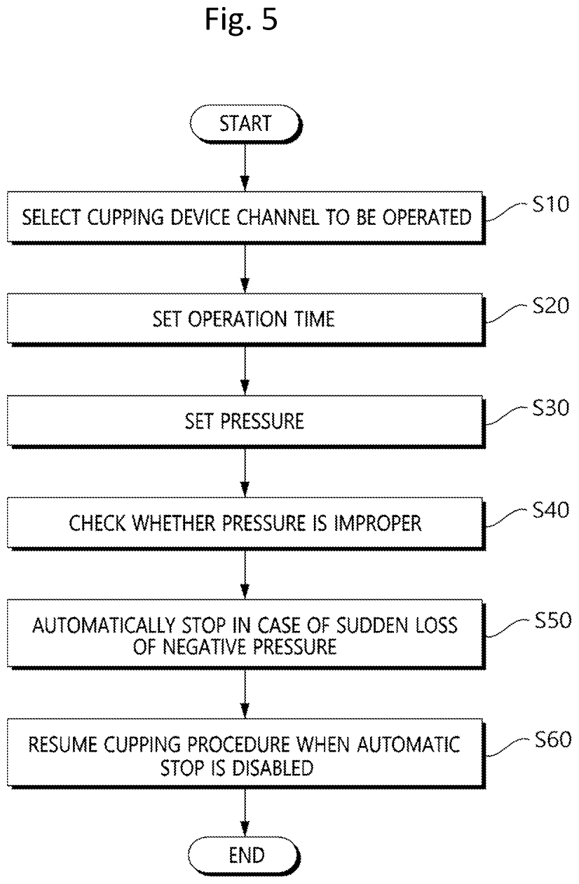

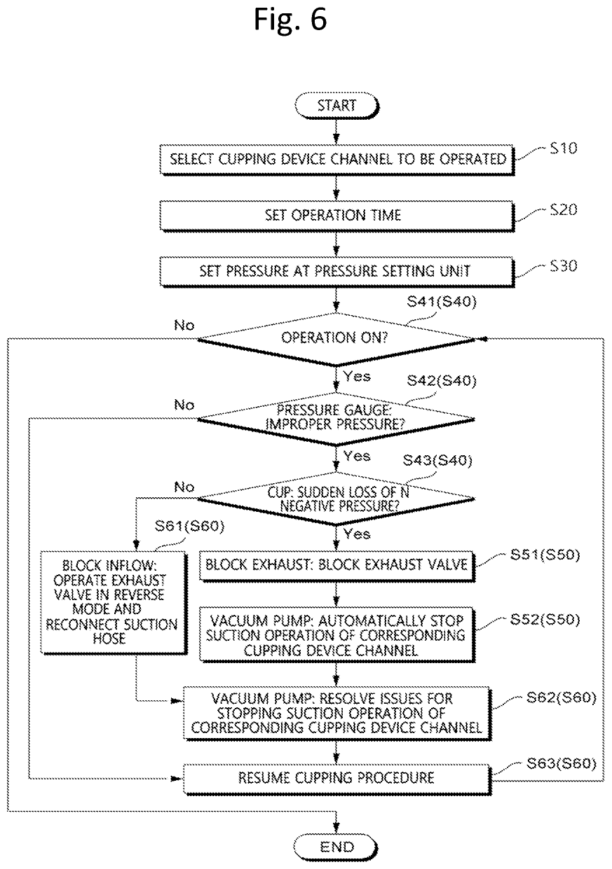

FIGS. 5 and 6 are flowchart for schematically explaining a method of using an electrical cupping system according to another embodiment of the present invention.

A method of using a cupping device system according to the present invention is a method of using an electrical cupping device system which uses the plurality of scalp cupping devices 10 described above, each including a negative pressure-controlling main cup 100 made of a plastic material and a body contact auxiliary cup 200 inserted into and coupled to an open lower portion of the main cup 100 and made of a silicone material relatively flexible compared to the main cup 100 to increase the sealing force when in contact with the body.

In addition, as shown in the drawings, the method of using an electronic cupping system according to an embodiment of the present invention includes selecting a cupping device channel to be operated (S10), setting an operation time (S20), setting a pressure (S30), checking whether the pressure is improper (S40), automatically stopping in case of a sudden loss of negative pressure (S50), and resuming the cupping procedure when the automatic stop is disabled (S60).

In more detail, the selecting of the cup device channel to be operated (S10) may be a step of selecting at least one cupping device channel to be operated from among the plurality of scalp cupping devices 10.

In addition, the setting of the operation time (S20) may be a step of setting an operation time of the selected cupping device channel by using the timer switch 311 of the control unit 310 provided in the negative pressure control means 300.

In addition, the setting of the pressure (S30) may be a step of setting a pressure of the selected cupping device channel using the pressure control device 340 provided in the negative pressure control means 300.

In addition, the checking of whether the pressure is improper (S40) may include checking a button of the on/off operation unit 330 provided in the negative pressure control means 300 and, when the operation is in on state (S41), checking whether a pressure gauge provided in the pressure control device 340 indicates improper pressure (S42).

In addition, the automatic stopping in case of a sudden loss of negative pressure (S50) may include checking whether the pressure gauge provided in the pressure control device 340 indicates improper pressure, and when there is a sudden loss of negative pressure (S43), blocking the exhaust valve 111 of the selected cupping device channel (S51) to automatically stop the suction operation (S52).

Further, the resuming of the cupping procedure when the automatic stop is disabled (S60) may include, when there is no sudden loss of negative pressure by checking whether the pressure gauge provided in the pressure control device 340 indicates improper pressure, operating in a reverse mode (S61) in which the exhaust valve 111 and the suction hose 301 is disconnected to block the air from flowing into the cup by the exhaust valve 111, resolving issues for stopping the suction operation by automatically turning off the cupping device channel of the disconnected suction hose 301 and reconnecting the suction hose 301 to turn on the cupping channel, and resuming the cupping procedure (S63).

In this case, according to the embodiment of the present invention, when the exhaust is blocked after the issues for stopping the suction operation of the vacuum pump for the selected corresponding cupping device, an exhaust blocking state is released using the exhaust blocking release button 112a, and when the inflow is blocked, an inflow blocking state may be released using the negative pressure release button 113 or the suction operation of the vacuum pump for the selected corresponding cupping device channel.

Meanwhile, as shown in the drawings, in the method of using an electronic cupping device system according to the embodiment of the present invention, the steps following the step S40 of checking whether the pressure is improper may be repeatedly performed at regular intervals in the process of normal operation after the step S63 of resuming the cupping procedure.

In addition, in the method of using an electronic cupping device system according to the embodiment of the present invention, vacuum pressure may be directly applied to the cupping device and various accompanying operations may be controlled by the control unit 310 described above with reference in FIG. 4. Also, the cupping device 10 may be attached to the treatment site through the suction part 220 of the auxiliary cup 200 constituting the cupping device 10 and the vacuum pressure is applied by using the control unit to start the cupping procedure.

Additionally, the pressure control device 340 including the pressure gauge and the pressure setting unit, as shown in FIG. 4, is connected to the power control unit 20 and may automatically stop the suction operation or adjust the pressure to a set pressure value when a predetermined pressure level is exceeded, that is, may adjust the pressure such that the vacuum pressure started by the control unit 310 can be continuously maintained constant as a negative pressure inside the cupping device 10.

Further, according to the embodiment of the present invention, when the negative pressure release button 113 of the cupping device 10 operates to release the pressure, the negative pressure inside the cupping device 10 rapidly decreases, and in this case, the exhaust valve 112 of the exhaust pipe 111 may be made automatically closed in order to maintain the rapidly lost internal negative pressure of the cupping device 10.

On the other hand, even when the exhaust pipe 111 and the suction hose 301 of the cupping device 10 are disconnected, the exhaust valve 112 is automatically closed, thereby preventing the loss of negative pressure in the cupping device 10.

As described above, according to the present invention, the scalp cupping device, the electronic cupping device system using the same, and the method of using the cupping device are provided. The scalp cupping device includes an attachment suction member made of a heterogeneous silicone material and inserted into and coupled to a main body of the cupping device so that the cupping therapy can be performed on various areas where it is difficult to use a cupping device, including the scalp and joint areas. In addition, a user can easily adjust the negative pressure inside each cupping device even when using multiple cupping devices, and the exhaust pipe of the cupping device causing a loss of negative pressure from among the multiple cupping devices is automatically blocked to enable suitable control for the negative pressure loss of the cupping device, thereby providing the stable cupping therapy.

Although this document provides descriptions of preferred embodiments of the present invention, it would be understood by those skilled in the art that the present invention can be modified or changed in various ways without departing from the technical principles and scope defined by the appended claims.

1. A scalp cupping device (10) for discharging body waste by using a negative-pressure load obtaining suction force from the removal of the inner air thereof, comprising:
a negative pressure-controlling main cup (100) having a negative pressure adjustment means (110) provided at the upper side thereof so as to apply a negative-pressure load to the inside thereof; and
a body contact auxiliary cup (200) which is inserted into and coupled to the opened lower portion of the main cup (100), and which has, on the circumference of the bottom opening thereof, a contact portion for coming in contact with the body.

2. The scalp cupping device of claim 1, wherein:
the negative pressure-controlling main cup (100) is formed in the shape of a circular cup made of a plastic material with the opened lower portion, and
the body contact auxiliary cup (200) is formed in the shape of a circular cup made of a relatively flexible material compared to the main cup (100) so as to seal the contact portion with the negative pressure-controlling main cup (100) when inserted into and coupled to the negative pressure-controlling main cup (100).

3. The scalp cupping device of claim 2, wherein the body contact auxiliary cup (200) has a through hole (201) formed in the upper central portion thereof to transmit the negative-pressure load applied to the main cup (100) and has an outwardly protruding circumferential periphery of the bottom opening to come in contact with the body.

4. The scalp cupping device of claim 3, wherein the body contact auxiliary cup (200) comprises:
a coupling part (210) which is formed on the upper side thereof and made of a medium-hardness silicone material to prevent an inflow of external air by being in soft contact with the contact portion when coupled to the main cup (100);
a suction part (220) which is formed on the lower side thereof and which is made of a material having a relatively low hardness compared to the coupling part (210) and is formed by overlaying or connecting a low-hardness silicone material so as to be securely attached without gaps even in contact with the scalp and hair; and
a holder (230) which is formed in a hook shape between the coupling part (210) and the suction part (220) and made of a medium-hardness silicone material having a relatively high hardness compared to the suction part (220) so as not to be sucked into the main cup (100).

5. The scalp cupping device of claim 1, wherein the negative pressure adjustment means (110) provided in the main cup comprises:
an exhaust pipe (111) for discharging the inner air to generate a negative pressure;
an exhaust valve (112) connected to the exhaust pipe (111) and configured to discharge the inner air or block the air from flowing into the inside; and
a negative pressure release button (113) for releasing the negative pressure load applied to the inside, and
the negative pressure adjustment means (110) is provided between the main cup (100) and a suction hose (301) or between the suction hose (301) and a suction pipe (320).

6. The scalp cupping device of claim 5, wherein the negative pressure release button (113) further comprises a button press spring device and a rotational latch to keep the negative pressure release button always in a negative pressure release state in order to prevent damage caused by strong air pressure acting on the main cup to eyes, ears, soft skin, etc. of children or the weak.

7. The scalp cupping device of claim 5, wherein the exhaust pipe (111) comprises a blocking plate spring (111a) vertically formed therein; and a blocking plate (111b) formed in a disk shape at an end of the blocking plate spring (111a), and
the exhaust valve (112) comprises an exhaust blocking release button (112a) capable of moving the blocking plate (111b) in a downward vertical direction on the upper surface thereof in an area adjacent to the exhaust pipe (111).

8. The scalp cupping device of claim 7, wherein the blocking plate (111b) usually maintains an intermediate position (111c) inside the exhaust pipe (111) at the end of the blocking plate spring (111a), and moves up and down according to an air speed, wherein, when moving upward, the blocking plate (111b) seals the bottom surface of the exhaust pipe (111) to block an exhaust direction, and, when moving downward, seals an inlet hole formed on the main cup to maintain a vacuum.

9. The scalp cupping device of claim 7, wherein a blocking state in an exhaust direction is released using the exhaust blocking release button (112a) and a blocking state in an inflow direction is released by using the negative pressure release button (113) or by a suction operation of a vacuum pump.

10. An electronic cupping device system comprising:
a plurality of scalp cupping devices (10), each of which includes a negative pressure-controlling main cup (100) made of a plastic material and having a negative pressure adjustment means (110) composed of an exhaust pipe (111), an exhaust valve (112), and a negative pressure release button (113) provided on an upper side thereof so as to apply a negative-pressure load to the inside thereof, and a body contact auxiliary cup (200) inserted into and coupled to an opened lower portion of the main cup(100) and made of a silicone material relatively flexible compared to the main cup(100) to increase a sealing force when in contact with the body;
a negative pressure control means (300) configured to individually control the negative-pressure load applied to the inside of the plurality of scalp cupping devices (10); and
a power control unit (20) configured to control power supplied to the negative pressure control means (300).

11. The electronic cupping device system of claim 10, wherein the negative pressure control means (300) comprises:
a control unit (310) having a plurality of timer switches (311) in a one-to-one correspondence to the plurality of scalp cupping devices (10);
a plurality of suction pipes (320) connected in a one-to-one correspondence between the timer switches (311) of the control unit (310) and suction hoses (301) connected to the exhaust pipes (111) of the plurality of scalp cupping devices (10);
a plurality of on/off operation units (330) each of which is disposed between each exhaust pipe (111) constituting the plurality of scalp cupping devices (10) and the suction pipe (320) to control an on/off operation of the timer switch (311) of the control unit (310) with a remote button; and
a plurality of pressure control devices (340) connected in a one-to-one correspondence between the plurality of suction pipes (320) and the plurality of on/off operation units (330) so as to check and set a pressure value applied to each of the plurality of scalp cupping devices (10).

12. The electronic cupping device system of claim 11, wherein the negative pressure control means (300) automatically stop the suction operation when the pressure applied to each of the plurality of scalp cupping devices (10) exceeds a specific range.

13. A method of using an electronic cupping device system that uses a plurality of scalp cupping devices each of which includes a negative pressure-controlling main cup (100) made of a plastic material and a body contact auxiliary cup (200) inserted into and coupled to an opened lower portion of the main cup (100) and made of a silicone material relatively flexible compared to the main cup (100) to increase a sealing force when in contact with the body, the method comprising:
selecting at least one cupping device channel to be operated from among the plurality of scalp cupping devices (10);
setting an operation time of the selected cupping device channel by using a timer switch (311) of a control unit (310) provided in a negative pressure control means (300);
setting a pressure of the selected cupping device channel using a pressure control device (340) provided in the negative pressure control means (300);
checking a button of an on/off operation unit (330) provided in the negative pressure control means (300) and, when an operation is in on state, checking whether a pressure gauge provided in the pressure control device (340) indicates improper pressure;
when there is a sudden loss of negative pressure by checking whether the pressure gauge provided in the pressure control device (340) indicates improper pressure, blocking an exhaust valve (112) of the selected cupping device channel to automatically stop a suction operation; and
when there is no sudden loss of negative pressure by checking whether the pressure gauge provided in the pressure control device (340) indicates improper pressure, operating in a reverse mode and resolving issues for stopping the suction operation by reconnecting a disconnected suction hose to resume a cupping procedure.
